(19) United States
(12) Patent Application Publication
(10) Pub. No.: US 2021/0345910 A1
(43) Pub. Date: Nov. 11, 2021

(54) FURNITURE-INTEGRATED MONITORING SYSTEM AND LOAD CELL FOR SAME

(71) Applicants: UNIVERSITY OF PITTSBURGH-OF THE COMMONWEALTH SYSTEM OF HIGHER EDUCATION, PITTSBURGH, PA (US); THE UNITED STATES GOVERNMENT AS REPRESENTED BY THE DEPT. OF VETERANS AFFAIRS, WASHINGTON, DC (US)

(72) Inventors: JONATHAN L. PEARLMAN, PITTSBURGH, PA (US); RORY A. COOPER, GIBSONIA, PA (US); JONATHAN A. DUVALL, PITTSBURGH, PA (US); ANAND A. MHATRE, PITTSBURGH, PA (US)

(73) Assignees: UNIVERSITY OF PITTSBURGH-OF THE COMMONWEALTH SYSTEM OF HIGHER EDUCATION, PITTSBURGH, PA (US); THE UNITED STATES GOVERNMENT AS REPRESENTED BY THE DEPT. OF VETERANS AFFAIRS, WASHINGTON, DC (US)

(21) Appl. No.: 17/381,354

(22) Filed: Jul. 21, 2021

Related U.S. Application Data
(62) Division of application No. 15/544,109, filed on Jul. 17, 2017.
(60) Provisional application No. 62/105,809, filed on Jan. 21, 2015.

(51) Int. Cl. A61B 5/103; A61B 5/00; A61B 5/11; A61B 5/117; G01L 1/22; G01G 19/44; A61G 7/05; G01L 1/26; G16H 40/60

(52) U.S. Cl. CPC A61B 5/1036; A61B 2562/166; A61B 5/1115; A61B 5/117; A61B 5/447; G01L 1/2206; G01G 19/445; A61G 7/0527; G01L 1/26; G16H 40/60; A61B 5/002; A61B 5/1117; A61B 5/4815; A61B 5/6892; A61B 5/7275; A61B 2562/0252; A61G 2203/44; A61B 2560/0214; A61B 2562/0261; A61B 5/6891

(57) ABSTRACT

A load cell apparatus for use with a bed includes a housing having a top portion and a bottom portion, and a load cell device held by the bottom portion of the housing. The load cell device is structured to generate a signal having a magnitude that is proportional to a first force being applied to the load cell device. The load cell apparatus also includes a button member held by the housing in a manner wherein the button member is structured to engage the load cell device and apply the first force to the load cell device in response to a second force being applied to the top portion of the housing. Also, various systems for monitoring parameters such as weight, sleep quality, fall risk, and/or pressure sore risk that may incorporate such a load cell apparatus.

### CROSS-REFERENCE TO RELATED APPLICATIONS

This is a divisional application which claims priority from U.S. patent application Ser. No. 15/544,109, filed on Jul. 17, 2017, entitled “Furniture-Integrated Monitoring System and Load Cell for Same,” which is a 371 of PCT international Application No. PCT/US2016/013989, filed on Jan. 20, 2016, entitled “Furniture-Integrated Monitoring System and Load Cell for Same, which claimed priority under 35 U.S.C. § 119(e) from U.S. Provisional Patent Application No. 62/105,809, filed on Jan. 21, 2015, entitled “Furniture-Integrated Weight Measurement System and Load Cell for Same”, contents of which are incorporated herein by reference.

### BACKGROUND OF THE INVENTION

### 1. Field of the Invention

The present invention pertains to systems for physical and health related parameters, and in particular, to a monitoring system, such as a weight management system, that may be integrated within a piece of furniture such as a bed.

### 2. Description of the Related Art

The World Health Organization (WHO) indicates that worldwide obesity has nearly doubled since 1980 and is the fifth leading risk for global deaths. A similar trend has been evident in the United States, causing more than one-third (or 78.6 million) of the adult population and 17% of the youth to be obese. Obesity causes several health-related risks such as heart disease, stroke, type 2 diabetes, and certain types of cancer which makes it a leading cause of preventable death.

Among the 36% of the US population with a disability, obesity is both more prevalent and has greater consequences. For instance, obesity is known to exacerbate a large number of disabling conditions including physical, muscular-skeletal and mental disabilities. Hence, adults with disabilities are more prone to obesity-related chronic health conditions than those without. Unfortunately, very little attention has been given to such a matter of serious concern. It has been found that Americans with disabilities are less likely to engage in physical activities than those without disabilities, with only 15% achieving the recommended level of physical activity. People with lower limb impairments, specifically wheelchair users, have significantly increased obesity-related health risks due to the challenges of maintaining an activity lifestyle. Physical inactivity of wheelchair users with spinal cord injury (SCI) is related to cardiovascular diseases, high blood pressure, osteoarthritis, osteoporosis, pressure ulcers, urinary tract infections, and repetitive strain injuries in upper extremities. These secondary health problems cause a downward spiral of health and are major causes of mortality and morbidity in people with disabilities. This evidence indicates that a physically active lifestyle and healthy weight are critical for people with disabilities, especially wheelchair users, to avoid obesity-related health risks and enjoy a better quality-of-life.

Maintaining a healthy weight is a challenge for everyone. But when it comes to wheelchair users, there are a host of complex issues with regards to weight maintenance. Physical barriers to exercise and daily activities, attitudinal barriers towards disability and health, environmental barriers for participation, maintaining dietary needs over time, and type of disability are just some of them. While engaging in physical activity can be a considerable challenge for this population, monitoring of daily activities, physical health and weight, and providing useful feedback is one way to help them start or continue with physical activity.

The general population has a plethora of body monitoring devices ranging from simple pedometers to complex multi-sensor platforms for activity tracking. On the other hand, very few health-monitoring devices are available for wheelchair users. In addition to having limited access to activity monitors, wheelchair users face serious challenges with weight tracking. Weight-measuring devices appropriate for wheelchair users are both cumbersome and expensive, making them really only feasible in a clinic setting. Hospital and clinic-based scales such as roll-on, lift-based and bed scales are available for weight measurement, but have little applicability in the home for various reasons. Roll-on scales, for instance, require the person to be weighed with the wheelchair and then the person is transferred out to weigh the wheelchair separately, which requires assistance. Lift-based scales require assistance as well, since the wheelchair user must be transferred onto the lift's platform for weighing. Hospital-based bed scales are convenient for the in-patient population, but are not applicable for in-home use for several reasons: they cannot be integrated into a user's bed, they do not accommodate weight measurement for multiple people (e.g. husband/wife), they are expensive, and they do not provide the affordance of monitoring with mobile devices.

Considering the deficiencies of existing weight scale systems for people with disabilities and recognizing their need for a comprehensive weight management system, there is a need for the development of a bed-integrated scale for in-home use.

In addition, prior art bed integrated load cell based systems have been employed in a host of clinical studies for monitoring various other health parameters. Assessment of sleep quality is one of the major applications. In one study, described in Choi B H, Seo J W, Choi J M, Shin H B, Lee J Y, Jeong do U, et al., Non-constraining sleep/cake monitoring system using bed actigraphy. Medical & biological engineering & computing. 2007; 45(1):107-14. Epub 2006/12/06. doi: 10.1007/s11517-006-0134-1. PubMed PMID: 17146691.2007, load cells were placed under the bed legs to measure sleep efficiency and other sleep/wake related parameters by analyzing body movements in bed while asleep. For this method of monitoring sleep, the author coined the term “bed actigraphy”, which he compared to the lab-based gold standard method of sleep analysis polysomnography (PSG). In that study, bed actigraphy was found to be comparable with PSG and of clinical value. Along with sleep monitoring, such load cell based non-invasive systems can provide insights about sleep disorders. Monitoring of sleep-related breathing disorder, detection of lying position in bed, insomnia, circadian rhythm disorder, periodic limb movement disorder and restlessness are some of them. Measuring and tracking sleep patterns is significant as inefficient sleep is related to mortality and morbidity risks.

There is thus also a need for a bed integrated load cell based system for in-home use that may be used for sleep monitoring or to monitor the status of other health situations, such as rapid weight gain, that are symptoms of congestive heart failure and poor kidney function.

### SUMMARY OF THE INVENTION

In a first aspect, the present invention provides a load cell apparatus for use with a bed having a plurality of legs that includes a housing having a top portion and a bottom portion, and a load cell device held by the bottom portion of the housing. The load cell device is structured to generate a signal having a magnitude that is proportional to a first force being applied to the load cell device. The load cell apparatus also includes a button member held by the housing in a manner wherein the button member is structured to engage the load cell device and apply the first force to the load cell device in response to a second force being applied to the top portion of the housing.

The load cell apparatus may include a support mechanism, such as a flexible member provided between the top portion of the housing and the bottom portion of the housing or a series of flexible diaphragm or bushings held by the housing that is meant to eliminate off-axis forces being transferred through the body of the housing. That is, this design is tailored to ensure all of the force transferred from the bed leg passes directly into the tab load-cell. If force does pass through the housing (around the load cell) then it would lead to errors in measurement.

In another aspect, the present invention provides a system for determining a risk that a patient may develop pressure sores for use with a bed having a plurality of legs. The system includes a plurality of load cell apparatuses, each load cell apparatus being provided beneath a respective one of the legs. Each load cell apparatus is structured to generate a signal that is proportional to a weight on associated leg. The system further includes a processing apparatus coupled to each of the load cell apparatuses that is structured to: (i) receive the signal generated by each of the load cell apparatuses, (ii) determine periods of quiescence based on the received signals, and (iii) determine a risk factor for pressure sores based on the periods of quiescence.

In still another aspect, the present invention provides a system for predicting an imminent out-of-bed fall occurrence for use with a bed having a plurality of legs. The system includes a plurality of load cell apparatuses, each load cell apparatus being provided beneath a respective one of the legs. Each load cell apparatus is structured to generate a signal that is proportional to a weight on the associated leg. The system also includes a processing apparatus coupled to each of the load cell apparatuses that is structured to: (i) receive the signal generated by each of the load cell apparatuses, (ii) monitor a weight distribution on the legs based on the received signals, and (iii) determine that a fall is imminent based on the monitored weight distribution.

In yet another aspect, the present invention provides a system for determining which of a first user and a second user are in a bed having a plurality of legs. The system includes a plurality of load cell apparatuses, each load cell apparatus being provided beneath a respective one of the legs. Each load cell apparatus is structured to generate a signal that is proportional to a weight on the associated leg. The system also includes a processing apparatus coupled to each of the load cell apparatuses, the processing apparatus being structured to: (i) receive the signal generated by each of the load cell apparatuses, and (ii) determine one of the following conditions based on the received signals: (1) none of the first user and the second user are in the bed, (2) only the first user is in the bed, (3) only the second user is in the bed, or (4) both the first user and the second user are in the bed. The processing apparatus may be further structured to determine a weight of the first user and a weight of the second user based on the determined condition and the received signals. It will be understood that the embodiments described herein that mention first and second users are not meant to cover just two users, but rather are meant to include two or more (i.e., multiple) users.

In still another aspect, the present invention provides a patient monitoring system that includes a plurality of bed monitors, wherein each bed monitor includes a bed having a plurality of legs, and a plurality of load cell apparatuses, each being provided beneath a respective one of the legs. Each load cell apparatus is structured to generate a signal that is proportional to a weight on the associated leg. The system further includes a processing apparatus coupled to each of the load cell apparatuses, the processing apparatus being structured to: (i) receive the signal generated by each of the load cell apparatuses, (ii) determine periods of quiescence based on the received signals, (iii) determine a risk factor for pressure sores based on the periods of quiescence, (iv) transmit the risk factor to at least one remote computing device, (v) monitor a weight distribution on the legs based on the received signals. (vi) determine that a fall is imminent based on the monitored weight distribution, (vii) generate a fall alarm in response to determining that a fall is imminent, and (viii) transmit the risk factor to the at least one remote computing device.

In another aspect, the data collected by the system could be combined with other data such as calorie intake, daily activity, etc. to provide a comprehensive health monitoring solution. The data could be used by the person who uses the bed or be passed to other family members (for example, to monitor whether grandma is sleeping, etc.) or clinicians to monitor behavior. Changes in weight and sleep habits are linked to some medical conditions, such as congestive heart failure (CHF). So, for example, a rapid weight change detected by the system could indicate water retention and trigger a medical alert. Also, weight measurements are important for medical dosing. Thus, frequent weight measurements related to medicine dosing may be used for alerting healthcare providers to symptoms such as congestive heart failure, kidney issues, etc. So, for example, if someone has an onset of CHF, a clinician could use the daily weight measurements to meter the dosage of lasixs.

In still another aspect, a monitoring system for use with a bed having a plurality of legs is provided. The system includes a plurality of load cell apparatuses each as described above, each load cell apparatus being structured to be provided beneath a respective one of the legs. The system also includes a processing apparatus coupled to each of the load cell apparatuses, the processing apparatus being structured to: (i) receive the signal generated by each of the load cell apparatuses, (ii) generate a second signal based on the signal generated by each of the load cell apparatuses, and (iii) cause the second signal to be communicated to a remote computer system having a remote database associated therewith.

### BRIEF DESCRIPTION OF THE DRAWINGS

FIG. 1 is a schematic diagram and FIG. 2 is a block diagram of a bed-integrated monitoring system for in-home use according to an exemplary, non-limiting embodiment of the disclosed concept:

FIGS. 3, 4 and 5 are exploded views of a load cell assembly of the monitoring system of FIG. 1 according to one particular exemplary embodiment;

FIG. 6 is an isometric view of a bottom housing portion of the load cell assembly of FIGS. 3, 4 and 5;

FIG. 7 is an isometric view of a mounting tray of the load cell assembly of FIGS. 3, 4 and 5;

FIG. 8 is an isometric view of a load cell cantilever piece of the load cell assembly of FIGS. 3, 4 and 5;

FIG. 9 is an isometric view of a flexible diaphragm member of the load cell assembly of FIGS. 3, 4 and 5;

FIGS. 10, 11 and 12 are isometric views of a button member of the load cell assembly of FIGS. 3, 4 and 5;

FIGS. 13 and 14 show the button member coupled to the flexible diaphragm member;

FIG. 15 is a side elevational view of a spherical ball of one particular exemplary embodiment of the load cell assembly of FIGS. 3, 4 and 5;

FIG. 16 is an isometric view of a control unit of the system of FIG. 1 according to one exemplary embodiment;

FIG. 17 is a schematic diagram of a patient monitoring system according to a further alternative exemplary embodiment of the disclosed concept;

FIGS. 18 and 19 are top and bottom isometric views, respectively, of a load cell assembly according to an alternative embodiment of the disclosed concept;

FIGS. 20 and 21 are bottom isometric and cross sectional views, respectively, of a load cell assembly according to another alternative embodiment of the disclosed concept;

FIG. 22 is a schematic diagram of a bed-integrated monitoring system for in-home use according to an alternative exemplary embodiment of the disclosed concept;

FIG. 23 is a schematic diagram of a master load cell assembly of the monitoring system of FIG. 22;

FIG. 24 is a schematic diagram of a slave load cell assembly of the monitoring system of FIG. 22;

FIG. 25 is a top-level schematic illustrating a machine learning algorithm implemented in the monitoring system described herein according a particular exemplary embodiment;

FIG. 26 is a flowchart illustrating operation of the algorithm of FIG. 25;

FIG. 27 is a top-level schematic illustrating a machine learning algorithm implemented in the monitoring system described herein according another particular exemplary embodiment; and

FIG. 28 is a flowchart illustrating operation of the algorithm of FIG. 27.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs.

As used herein, “directly coupled” means that two elements are directly in contact with each other.

As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body.

As employed herein, the statement that two or more parts or components “engage” one another shall mean that the parts exert a force against one another either directly or through one or more intermediate parts or components.

As employed herein, the term “number” shall mean one or an integer greater than one (i.e., a plurality).

As used herein, the terms “component” and “system” are intended to refer to a computer related entity, either hardware, a combination of hardware and software, software, or software in execution. For example, a component can be, but is not limited to being, a process running on a processor, a processor, an object, an executable, a thread of execution, a program, and/or a computer. By way of illustration, both an application running on a server and the server can be a component. One or more components can reside within a process and/or thread of execution, and a component can be localized on one computer and/or distributed between two or more computers.

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

The present invention will now be described, for purposes of explanation, in connection with numerous specific details in order to provide a thorough understanding of the subject invention. It will be evident, however, that the present invention can be practiced without these specific details without departing from the spirit and scope of this innovation.

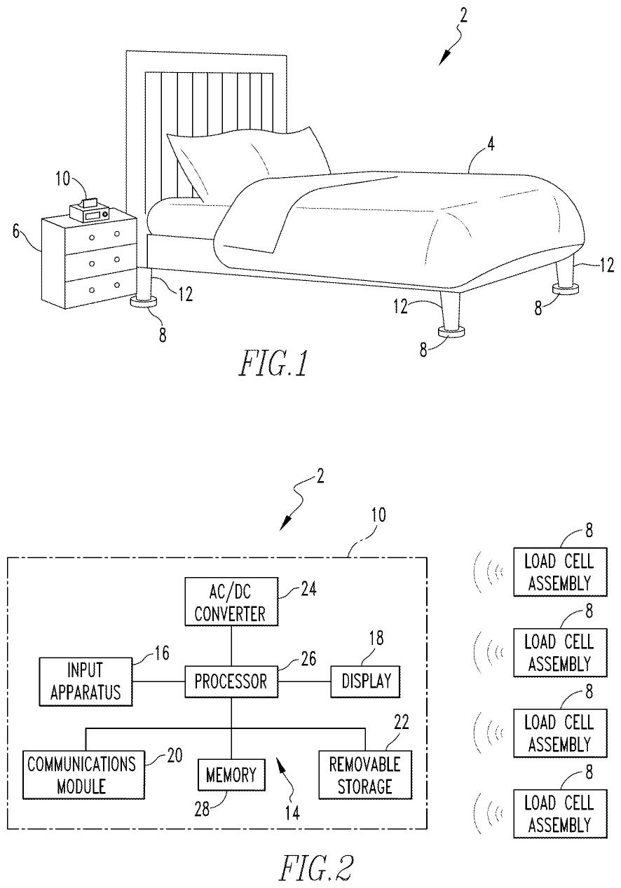

FIG. 1 is a schematic diagram and FIG. 2 is a block diagram of a bed-integrated monitoring system 2 for in-home use according to an exemplary, non-limiting embodiment of the disclosed concept that may be used for measuring and monitoring the weight of one or more individuals, such as one or more wheelchair users (in other exemplary embodiments described elsewhere herein, monitoring system 2 may also be used to monitor for other health and safety related conditions such as, without limitation, sleep, the potential for the development of pressure sores and/or the presence of conditions indicating that a fall is likely). As seen in FIG. 1, in the exemplary embodiment, monitoring system 2 is integrated in a home environment, such as a bedroom, that includes a bed 4 and a nightstand 6. Monitoring system 2 includes a plurality of (e.g., four) load cell assemblies 8 that are operatively coupled to a control unit 10. In the illustrated embodiment, each load cell assembly 8 is positioned beneath a respective one of the legs 12 of bed 4, and control unit 10 is positioned on nightstand 6. Each load cell assembly 8 is structured to measure the magnitude of a force that is being applied thereto by the respective leg 12 and to generate a signal indicative of that force. In addition, each load cell assembly 8 is in electronic communication with control unit 10. In the exemplary embodiment, each load cell assembly 8 is wirelessly connected to control unit 10 to provide such electronic communication (e.g., by having an onboard power source and wireless communications module such as a Bluetooth® module), although it will be understood that such electronic communication may also be provided via a wired connection. According to one aspect of the disclosed concept, control unit 10 is structured to receive each of the force signals from the load assemblies 8, which together are indicative of the weight present on bed 4, and to determine and display weight information relating to the weight of one or more users of bed 4. In the non-limiting exemplary embodiment, control unit 10 implements an algorithm that sums the weight data from each load cell assembly 8 and based thereon determines and displays the current weight of a user resting on bed 4. The weight data may be sampled periodically, e.g. every second, and control unit 10 has the capacity to log weight data for a period of time, such as one year.

Referring to FIG. 2, an exemplary embodiment of control unit 10 is shown. As seen in FIG. 2, control unit 10 includes a processor apparatus 14, an input apparatus 16 (such as one or more buttons or a touchscreen), a display 18 (such as a liquid crystal display (LCD)), a communications module 20 (which in the exemplary embodiment is a wireless communications module such as a Bluetooth® module and/or a WiFi module), a removable storage device 22 (such as a micro SD card) and an AC/DC converter 24 for providing DC power to control unit 10 from an AC source such as a wall outlet. A user is able to provide input into processor apparatus 14 using input apparatus 16, and processor apparatus 14 provides output signals to display 18 to enable display 18 to display information, such as weight information as described herein, to the user. In the illustrated, exemplary embodiment, processor apparatus 14 comprises a processor 26 and a memory 28. Processor 26 may be, for example and without limitation, a microprocessor (μP), a microcontroller, an application specific integrated circuit (ASIC), or some other suitable processing device, that interfaces with memory 28. Memory 28 can be any one or more of a variety of types of internal and/or external storage media such as, without limitation, RAM, ROM, EPROM(s), EEPROM(s), FLASH, and the like that provide a storage register, i.e., a machine readable medium, for data storage such as in the fashion of an internal storage area of a computer, and can be volatile memory or nonvolatile memory. Memory 28 has stored therein a number of routines that are executable by processor 26. One or more of the routines implement (by way of computer/processor executable instructions) at least one embodiment of the methods discussed herein for monitoring the weight or another health parameter relating to the user of bed 4.

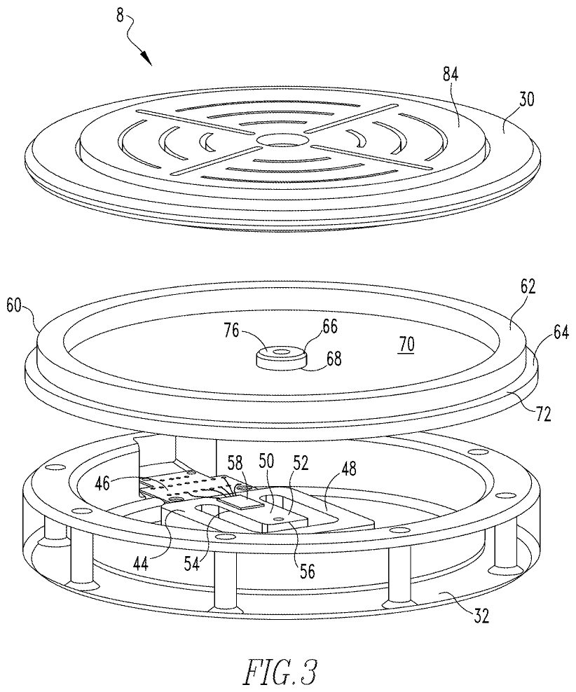

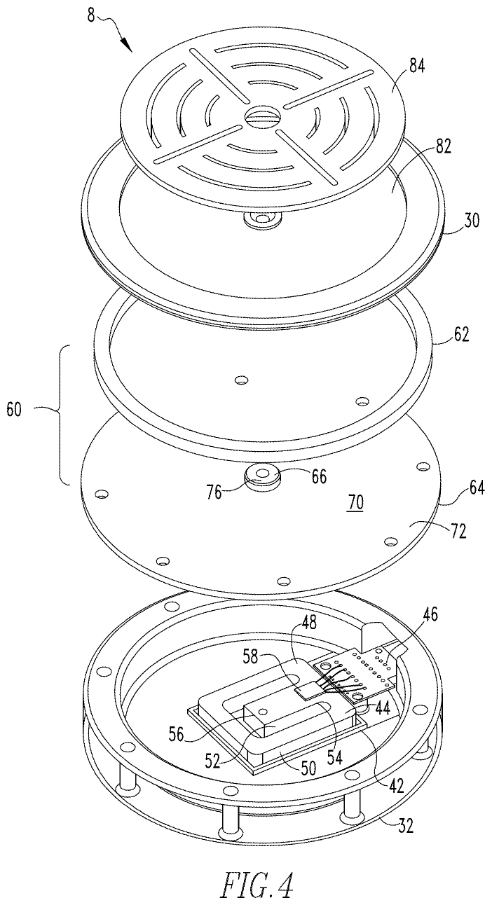

FIGS. 3, 4 and 5 are exploded views of load cell assembly 8 according to one particular exemplary embodiment of the disclosed concept. FIGS. 3 and 4 provide a top isometric perspective and FIG. 5 provides a bottom isometric perspective. As seen in FIGS. 3, 4 and 5, load cell assembly 8 in this embodiment includes a disk-shaped housing that includes a top housing portion 30 that is coupled to a bottom housing portion 32. As described herein, top housing portion 30 and bottom housing portion 32 are structured to house and support the various components of load cell assembly 8.

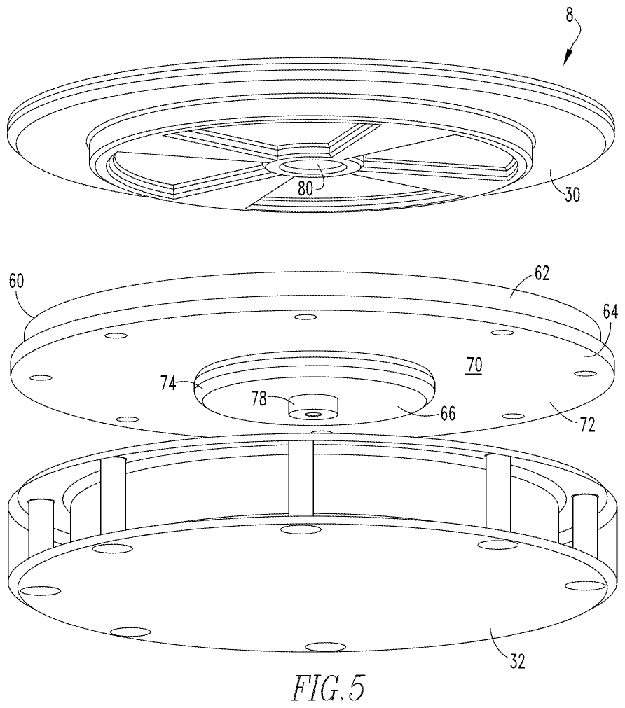

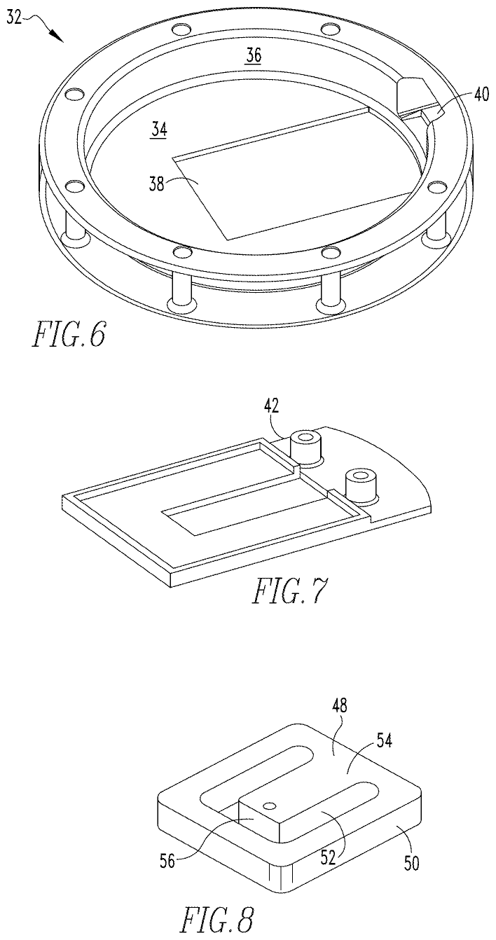

FIG. 6 is an isometric view of bottom housing portion 32. As seen in FIG. 6, bottom housing portion 32 includes a base member 34 having an outer wall 36 extending upwardly therefrom. Base member 34 includes a recessed pocket 38, and outer wall 36 includes a ledge portion 40 adjacent recessed pocket 38. In the exemplary embodiment, recessed pocket 38 is structured to receive and securely hold a mounting tray 42 as shown in FIG. 7. Mounting tray 42 is, in turn, structured to receive and hold a load cell 44 as seen in FIGS. 3 and 4. Furthermore, ledge portion 40 is structured to receive and hold a printed circuit board 46 (that includes thereon appropriate measurement, control and communications electronics) as shown in FIGS. 3 and 4. Load cell 44 and printed circuit board 46 are structured to, in cooperation with other parts of load cell assembly 8 described herein, generate the force indicative signals that are described elsewhere herein.

In the exemplary embodiment, load cell 44 includes a load cell cantilever piece 48 as shown in FIG. 8, which may be made of steel or any other suitable material. Load cell cantilever piece 48 includes an outer support frame portion 50 having a cantilever portion 52 extending therefrom and into an interior thereof. Cantilever portion 52 includes a proximal end 54 and a distal end 56. As seen in FIG. 3, load cell 44 further includes a number of strain gauges 58 that are provided on the surface of proximal end 54 of cantilever portion 52. In one particular exemplary embodiment, strain gauges 58 are provided on both the top and the bottom surfaces of proximal end 54. Strain gauges 58 are electrically connected to the electronic components provided on printed circuit board 46 such that measurements made by strain gauges 58 are communicated to printed circuit board 46 for further processing and/or transmission thereof as described herein.

In one particular exemplary embodiment, strain gauges 58 are soldered to form a full Wheatstone bridge with two strain gauges 58 on each side of proximal end 54 of cantilever portion 52 to compensate for temperature, to be highly sensitive to bending strain, and to avoid lead resistances and axial strain. The resulting voltage difference across the Wheatstone bridge is, in this embodiment, amplified by an amplifier device provided on printed circuit board 46 before the signals are sent to control unit 10 as described herein.

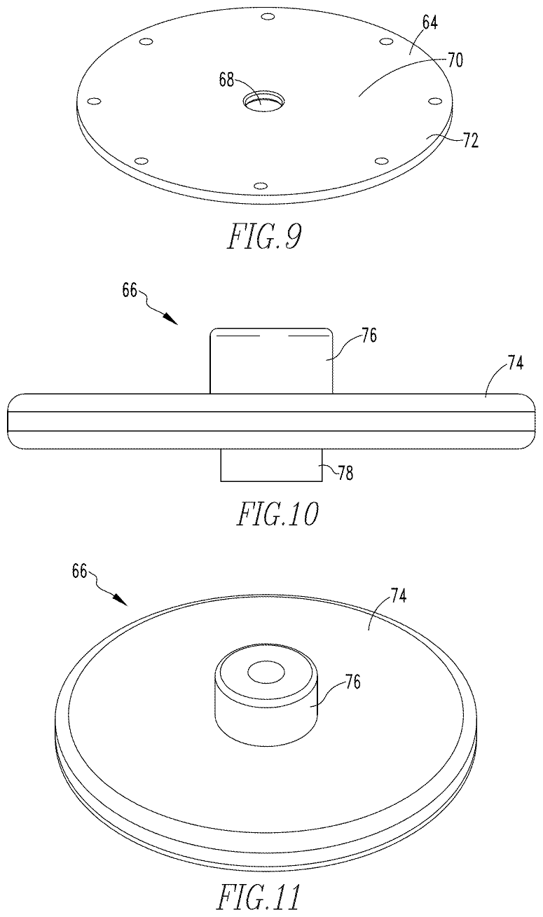

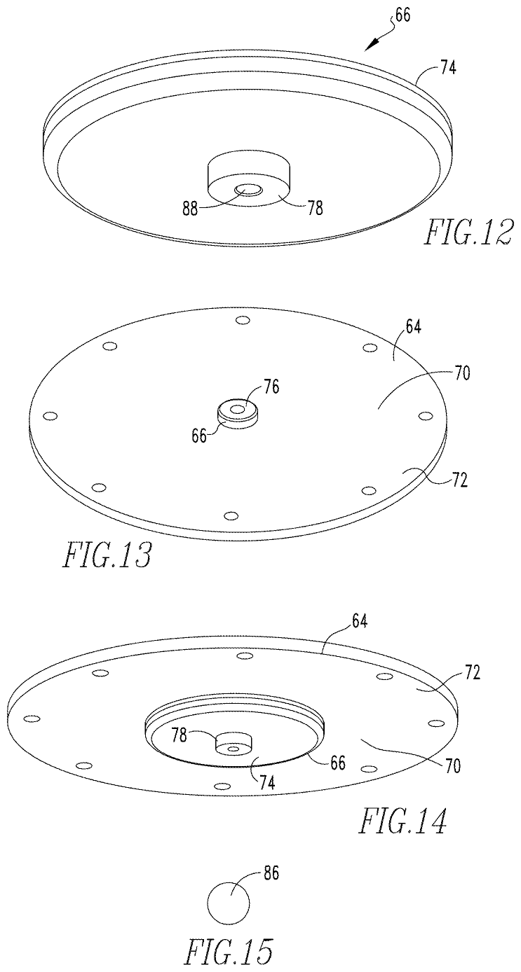

In addition, as seen in FIGS. 3, 4 and 5, load cell assembly 8 includes a load cell engagement assembly 60 that is structured to be provided between top housing portion 30 and bottom housing portion 32. Load cell engagement assembly 60 includes a ring member 62 provided on a top side of a flexible diaphragm member 64, and a button member 66 that is held and supported by flexible diaphragm member 64 as described herein. In the exemplary embodiment, ring member 62 is made of aluminum or another suitable rigid material, flexible diaphragm member 64 is made of rubber or another suitable flexible material such as, without limitation, silicone, and button member 66 is made of acrylonitrile butadiene styrene ABS or other rigid plastics. FIG. 9 is an isometric view of flexible diaphragm member 64 and FIGS. 10, 11 and 12 are side elevational, top isometric, and bottom isometric views, respectively, of button member 66 according to the exemplary embodiment.

Referring to FIGS. 3 and 9, flexible diaphragm member 64 includes a central aperture 68, an inner portion 70, and an outer edge portion 72. When load cell assembly 8 is assembled, outer edge portion 72 of flexible diaphragm member 64 is located beneath and outside of the outer perimeter of ring member 62 and forms a gasket member for providing a seal between top housing portion 30 and bottom housing portion 32, and inner portion 68 of flexible diaphragm member 64 is located within the inner perimeter of ring member 62 and provides a flexing member for transferring load forces to load cell 44 as described herein.

As seen in FIGS. 10, 11 and 12, button member 66 includes a central disk-shaped body 74, a top cylindrical button portion 76 provided on a top surface of body 74, and a bottom cylindrical button portion 78 provided on a bottom surface of body 74. In order to assemble engagement assembly 60, top cylindrical button portion 76 is inserted through a central aperture 68 of flexible diaphragm member 64 to form a sub-assembly as shown in FIGS. 13 and 14. Ring member 62 is then provided on the top surface of flexible diaphragm member 64 as shown in FIG. 3. Thereafter, to further assemble load cell assembly 8, engagement assembly 60 is provided between top housing portion 30 and bottom housing portion 32. When this is done, bottom cylindrical button portion 78 will engage distal end 56 of cantilever portion 52. In addition, as seen in FIG. 5, the bottom surface of top housing portion 30 includes a central circular recess 80 that is structured to receive top cylindrical button portion 76 therein when load cell assembly 8 is assembled. In addition, as seen in FIG. 4, in the illustrated embodiment, the top surface of top housing portion 30 is concave-shaped so as to accommodate a variety of types of bed legs. In addition, the top surface of top housing portion 30 includes a recessed portion 82 that receives a rubber disk member 84 therein to complete the assembly of load cell assembly 8. Rubber disk member 84 is engraved so as to allow a user to position bed legs 12 coaxially to avoid any off-centered loading of load cell assembly 8 during installation. In one particular embodiment, top housing portion 30 is painted a bright color for the color to show through the engravings in rubber disk member 84.

In one particular exemplary embodiment, a spherical steel ball 86 is provided within a central bore 88 provided in bottom cylindrical button portion 78. In this embodiment, it is spherical steel ball 86 that directly engages distal end 56 of cantilever portion 52. In this configuration, spherical steel ball 86 provides a single point of loading of distal end 56 of cantilever portion 52. Thus, the configuration of load assembly 8 as described provides for single point force transmission from leg 12 to load cell 44 centrally.

In operation, when a force is applied to top housing portion 30 through rubber disk member 84, that force is transferred to top cylindrical button portion 76. That force causes flexible diaphragm member 64 to flex such that the force is then transferred to distal end 56 of cantilever portion 52 through bottom cylindrical button portion 78 (and, in the example embodiment, through spherical steel ball 86). When such force is applied to distal end 56 of cantilever portion 52, strain gauges 58 will make measurements indicative thereof that are provided to the electronics on printed circuit board 46. As described elsewhere herein, in the exemplary embodiment, the force signals generated based upon such measurements may be wirelessly transmitted to control unit 10 for use thereby as described herein.

FIG. 16 is an isometric view of control unit 10 according to one particular, non-limiting exemplary embodiment. Control unit 10 includes a housing 88 that houses the various components shown in FIG. 2. As noted elsewhere herein, control unit 10 is structured to display weight information instantly to the user of bed 4 using display 18. In the exemplary embodiment, processor apparatus 14 sums weight data from each load cell assembly 8 and converts the result into a known weight format for display on display 18. In the illustrated embodiment, housing 88 is provided with a dock 94 docking a mobile phone or similar device.

According to one alternative exemplary embodiment, monitoring system 2 is configured to determine a risk that a user of the bed will develop pressure sores. Such an implementation is of particular use for individuals with spinal cord injuries and/or other mobility and/or sensory impairments. In order for such people to avoid pressure sores or other complications, it is necessary for them to change their body position in bed after a certain period of time. Thus, in this exemplary embodiment, processor apparatus 14 includes one or more routines that are structured to receive the signals from each of the load cell assemblies 8 that are proportional to the weight on the leg 12 that is associated with the load cell assembly 8 and, from those signals, determine periods of quiescence (i.e., no motion). In the exemplary embodiment, such periods of quiescence are determined by substantially static (substantially unchanging) force measurements (e.g. less than 5 lbs.) over a predetermined duration of time, such as 30 minutes, while the user is in bed. In one embodiment, this would be accomplished by monitoring force measurements on each load cell assembly 8, and determining if it changes over a threshold (such as 5 lbs). Alternatively, center of pressure could be determined by identifying the average location of the weight and monitoring whether that average location moves by a certain percentage or distance (assuming the bed size is known). In addition, the routines are structured to log such determined periods of quiescence and, based on the amount, duration, and/or frequency of such periods, determine a risk factor indicating the likely risk that the user will develop pressure sores. That risk factor may, for example, be displayed on display 18 or sent to a remote alert system as described herein to indicate to the user or a caregiver of the user that the user should shift to another position.

According to another alternative exemplary embodiment, monitoring system is configured to predict an out-of-bed fall occurrence before it occurs. In this exemplary embodiment, processor apparatus 14 includes one or more routines that are structured to receive the signals from each of the load cell assemblies 8 that are proportional to the weight on the leg 12 that is associated with the load cell assembly 8 and, from those signals, monitor the weight distribution among the load cell assemblies 8. In this embodiment, changes in such weight distribution are monitored for conditions that indicate that a fall out of bed 4 is imminent, such as the center of pressure of an occupant of bed 12 approaching the edge of bed 12.

In still another alternative embodiment, monitoring system 2 may be configured to both determine a risk that a user of the bed will develop pressure sores as just described and predict an out-of-bed fall occurrence before it occurs.

According to still another alternative exemplary embodiment wherein bed 4 is used by multiple (e.g., two) users, monitoring system 2 is configured to determine at any particular time which user(s) is on the bed, and using that information to monitor the weight of each user over time. In this embodiment, processor apparatus 14 is provided with a machine teaming algorithm that has been trained in advance using certain known “truth” data to be able to segregate the data collected by the load cell assemblies 8 as described herein into one of the following four categories: (1) no users on bed 4, (2) user 1 only is on bed 4, (3) user 2 only is on bed 4, and (4) both user 1 and user 2 are on bed 4. In the exemplary embodiment, the machine learning algorithm employs a Naïve Bayes classifier having a support vector machine that is trained in advance with known “truth” data, and the classifier is used to segregate the data into the four categories just described and to thereafter determine individual weights of the two users. The exemplary embodiment operates as follows. First, during a setup stage, each user (user 1 and user 2 in the present example) will set up a profile in processor apparatus 14 and then sit/rest on their side of the bed one at a time so that readings can be taken from each of the load cell assemblies 8. Next, during an operational stage, processing apparatus 14 will periodically receive and record weight data from each of the load cell assemblies 8 and determine the times at which the readings from the load cell assemblies 8 change. Processing apparatus 14 will then use the trained Naïve Bayes classifier to analyze the recorded data so that it will be able to segregate the data for any particular time into one of the four categories identified above. In addition, based on the categorization, processing apparatus 14 is able to determine and record individual weights for each of the users. In addition to recording weight information for each of the users individually, this classification mechanism may also be used to determine and store other parameters for each of the users individually, such as, without limitation, sleep quality and motion related data such as periods of quiescence as described herein. In the exemplary embodiment, sleep quality is determined through activity, which is essentially the ratio of the amount of motion (in time) in bed normalized by the total time in bed.

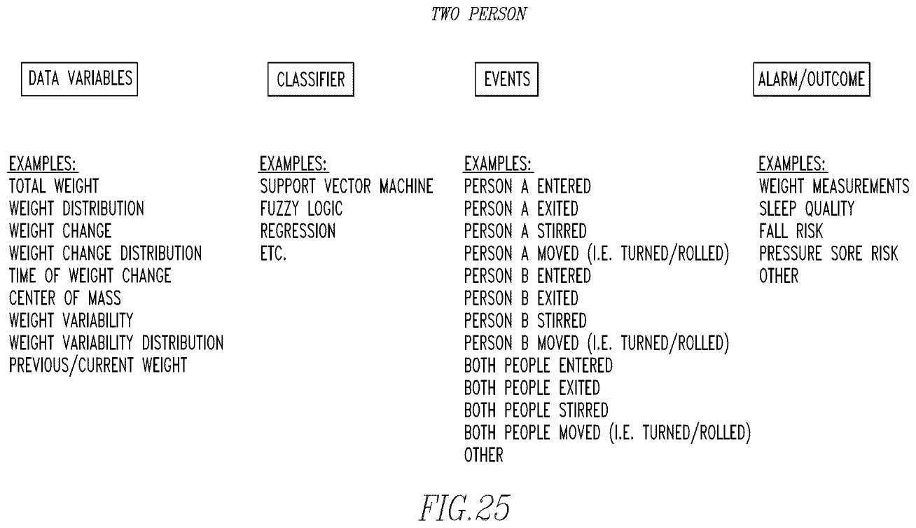

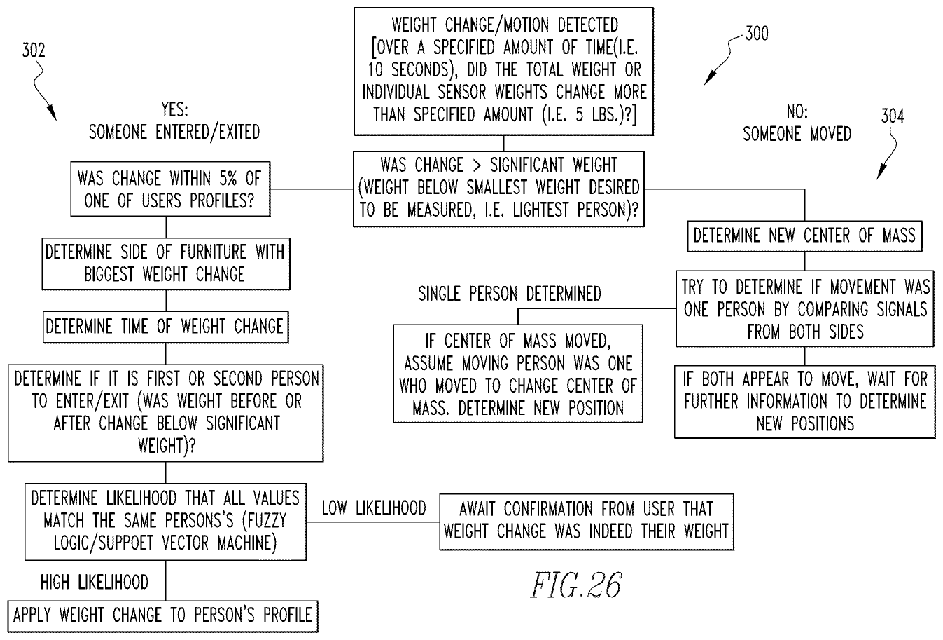

FIG. 25 is a top-level schematic illustrating an exemplary machine learning algorithm as just described implemented in monitoring system 2 according to another particular exemplary embodiment wherein weight, sleep quality, fall risk and pressure sore risk information, among others, may be monitored for two users. FIG. 25 illustrates the data variables, classifier, events, and alarm/outcome that may be implemented in such an embodiment. In addition, FIG. 26 is a flowchart 300 illustrating operation of such a machine learning algorithm according to one particular implementation. As seen in FIG. 26, operation of the machine learning algorithm includes a first branch 302 that is executed when one of the users enters or exits bed 4, and a second branch 304 that is executed when, instead, it is determined that a user of bed 4 has moved.

It will be understood that the embodiment described above that mentions two users (user 1 and user 2) is not meant to cover just two users, but rather may also include two or more (i.e., multiple) users. Thus, a third profile could be determined and used. For example, a child could climb on the bed and be weighed. Alternatively, a child and their parents could be on the bed together and the system could determine all of their individual weights simultaneously. The system may thus be used to help determine which, if any, of the people/pets/etc. are on the bed individually or together, and then keep a “diary/log” for each which includes weight, sleep behavior, etc.

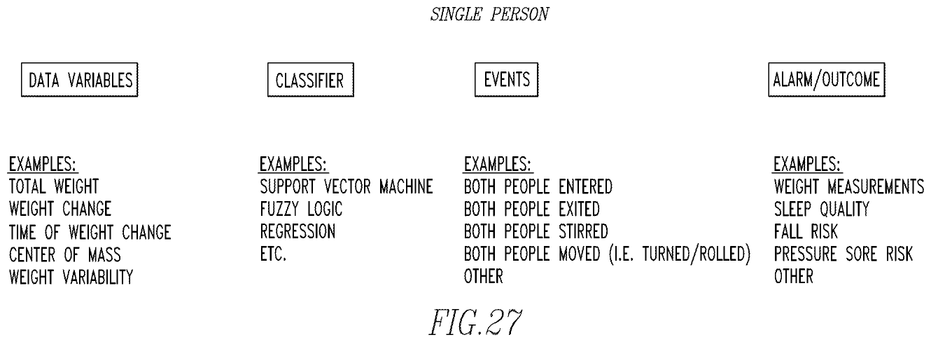

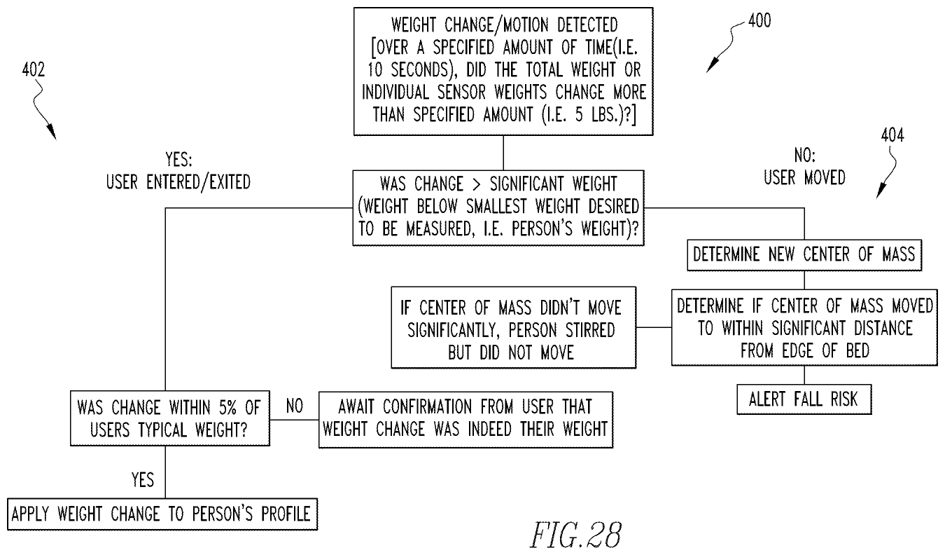

FIG. 27 is a top-level schematic illustrating a machine learning algorithm implemented in monitoring system 2 according to still another particular exemplary embodiment wherein weight, sleep quality, fall risk and pressure sore risk information, among others, may be monitored for a single user. FIG. 27 illustrates the data variables, classifier, events, and alarm/outcome that may be implemented in such an embodiment. In addition, FIG. 28 is a flowchart 400 illustrating operation of such a machine learning algorithm according to one particular implementation. As seen in FIG. 26, operation of the machine learning algorithm includes a first branch 402 that is executed when the user enters or exits bed 4, and a second branch 304 that is executed when, instead, it is determined that the user of bed 4 has moved.

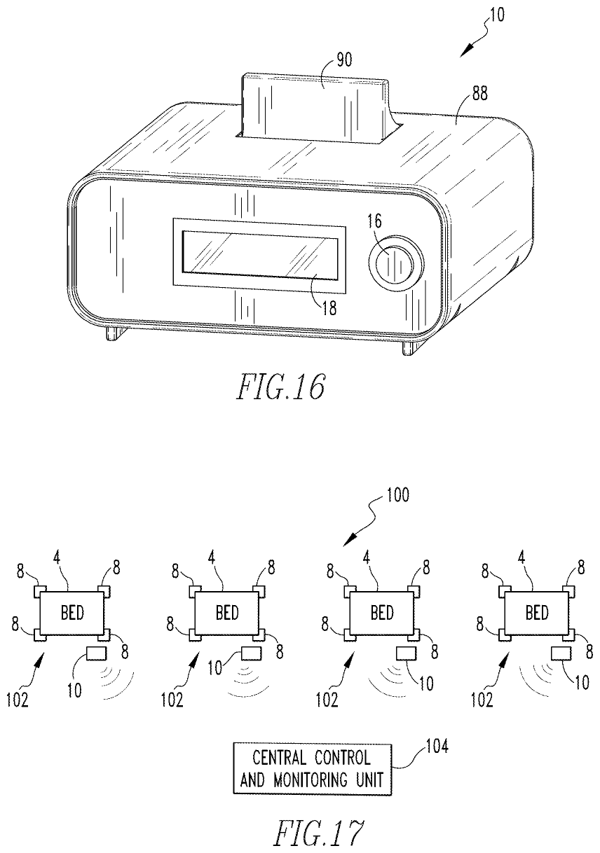

FIG. 17 is a schematic diagram of a patient monitoring system 100 according to a further alternative exemplary embodiment of the disclosed concept. Patient monitoring system 100 may be employed in a clinical setting, such as a hospital or nursing home, to monitor various patients. As seen in FIG. 17, patient monitoring system 100 includes a plurality of bed monitors 102, wherein each bed monitor 102 includes a bed 4, a plurality of load cell apparatuses 8 as described herein, and a control unit 10 as described herein (including a real time clock for time stamping collected data). Each bed monitor 102 is structured to operate as described herein. In particular, the control unit 10 of each bed monitor 102 is structured to receive the signal generated by each of the load cell assemblies 8 associated therewith. Further, the control unit 10 of each bed monitor 102 is also structured to determine periods of quiescence based on the received signals, determine a risk factor for pressure sores based on the periods of quiescence, monitor a weight distribution on the associated bed 4 based on the received signals, determine that a fall is imminent based on the monitored weight distribution, and generate a fall alarm in response to determining that a fall is imminent. Patient monitoring system 100 further includes a remote computing device in the form of central control and monitoring unit 104, which may be located at, for example without limitation, a nurse's station. The bed monitors 102 are, in the illustrated exemplary embodiment, each structured to transmit (in a wired or wireless manner) the determined risk factor and fall alarm to the central control and monitoring unit 104 so that a caregiver can be made aware of such conditions. In an alternative embodiment, each bed monitor 102 may be structured to transmit (in a wired or wireless manner) the signals generated by the associated load assemblies 8 to central control and monitoring unit 104 which then centrally determines the risk factor and the fall alarm as described herein for each bed monitor 102 as appropriate.

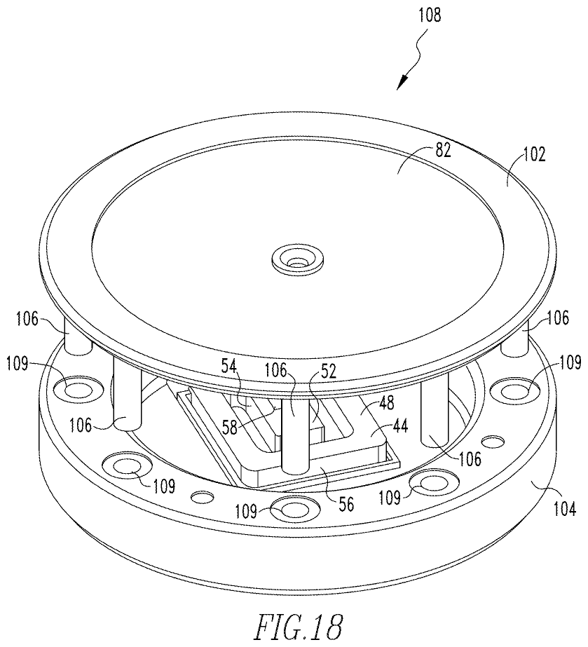

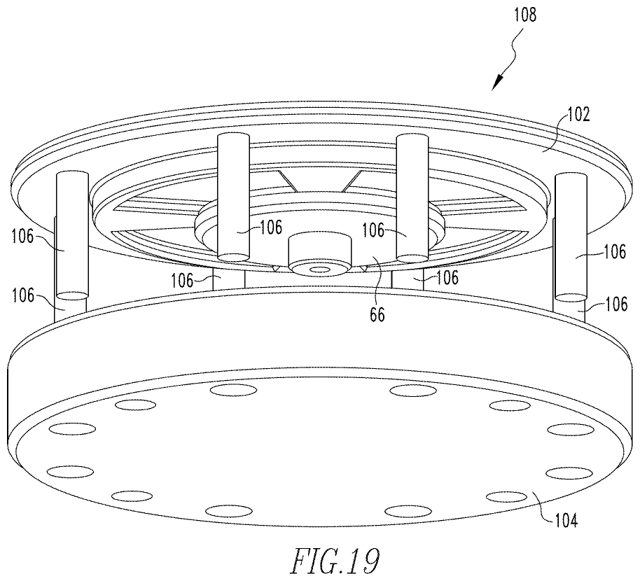

FIGS. 18 and 19 are top and bottom isometric views, respectively, of a load cell assembly 108 according to an alternative embodiment of the disclosed concept. Load cell assembly 108 may be substituted for load cell assembly 8 in the various embodiments described herein. Load cell assembly 108 includes a disk-shaped housing that includes a top housing portion 102 that is similar in structure to top housing portion 32 that is coupled to a bottom housing portion 104. Top housing portion 102 and bottom housing portion 104 of the present alternative embodiment are structured to house and support the various components of load cell assembly 108, which include a load cell 44, a printed circuit board 46 (not shown), and a button member 66 as described elsewhere herein. As seen in FIG. 19, button member 66 in this embodiment is attached to and held by the bottom surface of top housing portion 102. Button member 66 includes bottom cylindrical button portion 78 for engaging load cell 44 as described herein. As seen in FIGS. 18 and 19, top housing portion 102 includes a plurality of 10 members 106 that extend from the bottom surface thereof. In addition, bottom housing portion 104 includes a plurality of channel members 109 that are each structured to receive and hold a respective pin member 106 in a manner which holds the pin member 106 in place horizontally but allows for vertical movement. In one embodiment, each channel member 109 comprises a linear bushing member. In another embodiment, each channel member comprises a flexible diaphragm member. In this embodiment, the structure including pin members 106 and channel members 109 prevent top housing portion 102 from tipping in the event of off-center loading while at the same time transferring the load applied to top housing portion 102 to load cell 44.

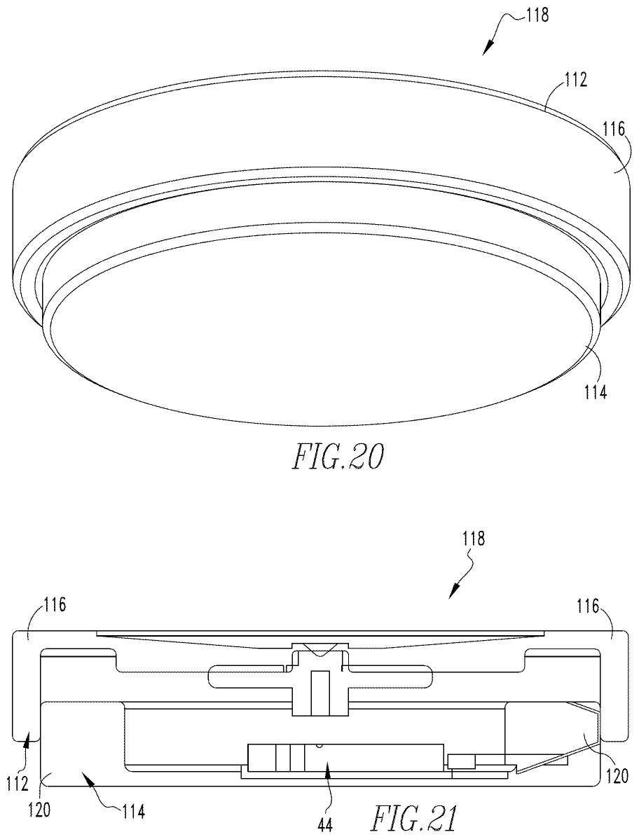

FIGS. 20 and 21 bottom isometric and cross sectional views, respectively, of a load cell assembly 118 according to another alternative embodiment of the disclosed concept. Load cell assembly 118, like load cell assembly 108, may be substituted for load cell assembly 8 in the various embodiments described herein. Load cell assembly 118 includes a disk-shaped housing that includes a top housing portion 112 that is similar in structure to top housing portion 32 that is coupled to a bottom housing portion 114. Top housing portion 102 and bottom housing portion 104 of the present alternative embodiment are structured to house and support the various components of load cell assembly 118, which include a load cell 44, a printed circuit board 46 (not shown), and a button member 66 as described elsewhere herein. As seen in FIG. 21, button member 66 in this embodiment is attached to and held by the bottom surface of top housing portion 112. As also seen in FIG. 21, top housing portion 112 includes an outer ring or flange member 116 that extend from the top portion thereof. Top housing portion 112 and bottom housing portion 114 are structured such that outer wall 120 of bottom housing portion 114 engages the flange member 116 but allows relative vertical movement between the 2 components to enable button member 66 to engage the load cell 44, while at the same time preventing top housing portion 112 from tipping in the event of off-center loading.

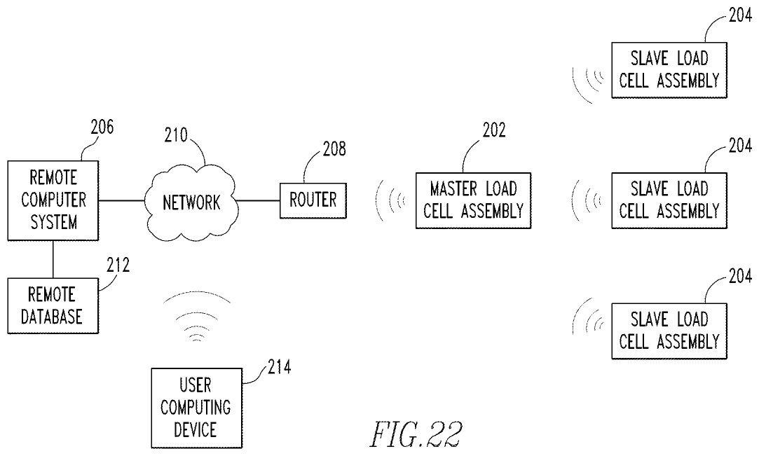

FIG. 22 is a schematic diagram of a bed-integrated monitoring system 200 for in-home use according to an alternative exemplary embodiment of the disclosed concept that may be used for measuring and monitoring the weight of one or more individuals, such as one or more wheelchair users (in other exemplary embodiments, monitoring system 200 may also be used to monitor for other health and safety related conditions such as, without limitation, sleep, the potential for the development of pressure sores and/or the presence of conditions indicating that a fall is likely). Like monitoring system 2, monitoring system 200 is integrated in a home environment, such as a bedroom, that includes a bed 4 as described herein (not shown in FIG. 22). Monitoring system 200 is structured as a master/slave system and includes a master load cell assembly 202 and a plurality of (e.g., three) slave load cell assemblies 204 that are operatively coupled to master load cell assembly 202. As described in greater detail herein, master load cell assembly 202 is structured to include most if not all of the functionality of control unit 10 (with the exception of displaying information in the exemplary embodiment).

Each load cell assembly 202, 204 is structured to be positioned beneath a respective one of the legs 12 of bed 4 (in the same manner as shown in FIG. 1 in connection with load cell assembly 8). In the exemplary embodiment, master load cell assembly 202 and each slave load cell assembly 204 include a load cell 44 and a housing structure according to any of the embodiments described herein (e.g., that of load cell assembly 8, load cell assembly 108 or load cell assembly 118). Each load cell assembly 202, 204 is structured to measure the magnitude of a force that is being applied thereto by the respective leg 12 and to generate a signal indicative of that force. In the present embodiment, each slave load cell assembly 204 is in electronic communication with master load cell assembly 202. In the exemplary embodiment, each slave load cell assembly 204 is wirelessly connected to master load cell assembly 202 to provide such electronic communication (e.g., by having an onboard power source and wireless communications module such as a Bluetooth® module), although it will be understood that such electronic communication may also be provided via a wired connection. According to one aspect of the disclosed concept, master load cell assembly 202 is structured to receive each of the force signals from slave load assemblies 204, which together with the force measurement made by master load cell assembly 202 are indicative of the weight present on bed 4.

Master load cell assembly 202 is structured to determine weight information relating to the weight of one or more users of bed 4 and to communicate that weight information to a remote computer system 206. In the exemplary embodiment, such remote communication is performed by first transmitting the information wirelessly to a router 208, such as a Wi-Fi router, which then transmits the information to remote computer system 206 through a network 210, such as the Internet. Alternatively, master load cell assembly 202 may include a communications module 20 capable of broadband wireless communications to enable data to be transmitted therefrom to remote computer to a 6 using a cellular data network. A remote database 212 is associated with remote computer system 206 for storing the weight (and possibly other) information of a number of users of monitoring system 200. That information may then be selectively provided to the user by transmitting that information to a user computing device 214, such as a smart phone, tablet or PC, though network 210 in a known manner.

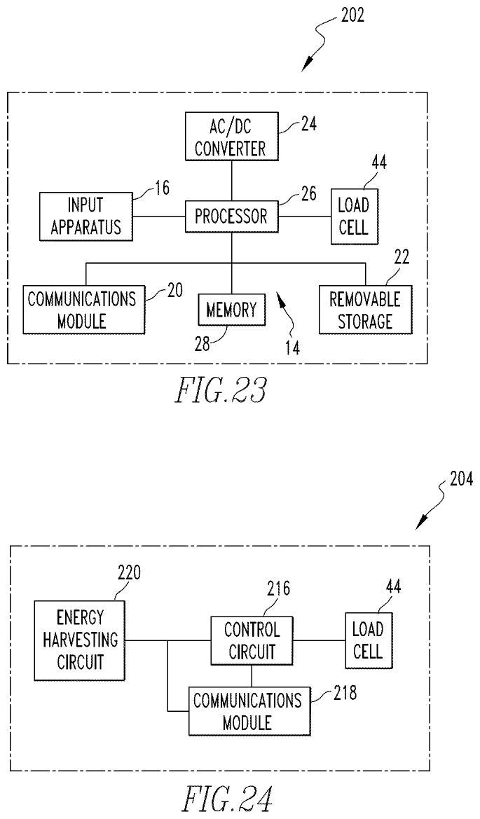

FIG. 23 is a schematic diagram of master load cell assembly 202 according to one non-limiting exemplary embodiment. As seen in FIG. 23, master load cell assembly 202 includes many of the same components as monitoring unit 10, and like components are labeled with like reference numerals. In addition, as mentioned above, master load cell assembly 202 also includes load cell 44 as described herein. In this embodiment, communications module 20 includes a Bluetooth® module for communicating with each slave load assembly 204, and a Wi-Fi module for communicating with router 208. Also in this embodiment, master load cell assembly 208 is powered by being plugged into a wall outlet or similar AC source as described elsewhere herein.

FIG. 24 is a schematic diagram of slave load cell assembly 204 according to one non-limiting exemplary embodiment of the disclosed concept. Slave load cell assembly 204 includes a control circuit 216, load cell 44 as described herein, and a communications module 218, which in the exemplary embodiment is a Bluetooth® module. In addition, in the illustrated embodiment, rather than being powered by an AC source such as a wall outlet or by an on-board battery, each slave load cell assembly 204 is provided with energy harvesting circuitry 220 that is structured to harvest energy from the environment for powering slave load cell assembly 204. In one exemplary embodiment, energy harvesting circuitry 220 is a piezoelectric electric energy harvesting circuit that produces an electric charge when undergoing mechanical stress as a result of forces being applied to bed 4 as described herein. In another exemplary embodiment, energy harvesting circuitry 220 is a radio frequency (RF) energy harvesting circuit which collects ambient or transmitted RF signals to generate power. In particular, in this exemplary embodiment, energy harvesting circuitry 220 is structured to convert ambient or transmitted RF energy that is received by an antenna thereof from an AC voltage to a DC voltage which is then used to provide operating power for slave load cell assembly 204. Such energy harvesting technology is well known in the art and is described in, for example, and without limitation, U.S. Pat. Nos. 6,289,237, 6,615,074, 6,856,291, 7,057,514, and 7,084,605, the disclosures of which are incorporated herein by reference. In the exemplary embodiment, such RF energy harvesting circuitry comprises a matching circuit/charge pump combination that is coupled to an antenna 22. An exemplary system that could be used is the P1110 RF power harvesting system by Powercast Corporation.

In still another alternative embodiment, monitoring system 2 as described herein may be structured to communicate with remote computer system 206 of FIG. 22 as described herein to provide similar remote access functionality to that described in connection with monitoring system 200. In addition, in monitoring system 2, each load cell assembly 8 could be provided with energy harvesting circuitry 220 as described above in order to provide power thereto in lieu of using a battery.

Furthermore, in embodiments which employ remote computer system 206, such embodiments may be further configured to enable remote computer 206 to determine one or any combination of the following in the manner described herein: (i) periods of quiescence and a risk factor for pressure sores, (ii) weight distribution on the bed legs and an indication that a fall is imminent based on the determined weight distribution, (iii) which of a first user and a second user are in the bed and based thereon make weight measurements over time.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

What is claimed is:

1. A monitoring system for predicting an imminent exit of an individual from a bed having a plurality of legs, comprising:
a plurality of load cell apparatuses, each of the load cell apparatuses including a housing and a load cell device held by the housing, the load cell device of each of the load cell apparatuses being structured to generate a signal having a magnitude that is proportional to a force being applied to the load cell device, each load cell apparatus being structured to be provided beneath a respective one of the legs; and
a computer system comprising a processing apparatus implementing a machine learning algorithm trained with certain truth data comprising in-bed weight distribution data and bed exit data, wherein the machine learning algorithm is structured and configured to: (i) obtain a number of weight distribution signals indicative of a weight distribution in the bed among the load cell apparatuses during a period of time, the number of weight distribution signals being based on the signals generated by the load cell apparatuses, and (ii) predict that the individual will exit the bed after the period of time but before actually exiting the bed based on the number weight distribution signals, wherein the computer system is structured and configured to generate an alarm indicating imminent exit from the bed in response to the machine learning algorithm predicting that the individual will exit the bed.

2. The monitoring system according to claim 1, wherein the processing apparatus is separate from each of the load cell apparatuses.

3. The monitoring system according to claim 1, wherein the load cell apparatuses include a master load cell apparatus and a number of slave load cell apparatuses, wherein the processing apparatus is part of the master load cell apparatus, wherein each slave load cell apparatus is structured to communicate the signal generated by the slave load cell apparatus to the master load cell apparatus.

4. The monitoring system according to claim 3, wherein each slave load cell apparatus includes energy harvesting circuitry for generating power for the slave load cell apparatus.

5. The monitoring system according to claim 4, wherein each energy harvesting circuitry comprises a piezoelectric energy harvesting circuit.

6. The monitoring system according to claim 4, wherein each energy harvesting circuitry comprises an RF energy harvesting circuit.

7. The monitoring system according to claim 1, wherein the computer system is a remote computer system located remotely from the load cell apparatuses, and wherein the remote computer system is structured to receive the signals generated by the load cell apparatuses and generate the number of weight distribution signals.

8. The monitoring system according to claim 1, wherein each of the load cell apparatuses includes one or more strain gauges.

9. The monitoring system according to claim 8, wherein in each of the load cell apparatuses the strain gauge is coupled to a cantilever piece.

10. The monitoring system according to claim 9, wherein in each of the load cell apparatuses the cantilever piece includes an outer frame member having a cantilever member extending therefrom, wherein the strain gauge is provided at a first end of the cantilever member and wherein a second end of the cantilever member is structured to be contacted by a button member of the load cell apparatus.

11. The monitoring system according to claim 1, wherein the machine learning algorithm has been previously trained using ground truth data comprising in-bed weight distribution data and bed exit data obtained from a plurality of test subjects.

12. A method of predicting an imminent exit of an individual from a bed having a plurality of legs and a plurality of load cell apparatuses, each of the load cell apparatuses being provided beneath a respective one of the legs and being structured to generate a signal having a magnitude that is proportional to a force being applied to the load cell apparatus, the method comprising:
obtaining a number of weight distribution signals indicative of a weight distribution in the bed among the load cell apparatuses during a period of time, the number of weight distribution signals being based on the signals generated by the load cell apparatuses;
providing the number of weight distribution signals to a machine learning algorithm trained with certain truth data comprising in-bed weight distribution data and bed exit data;
predicting in the machine learning algorithm that the individual will exit the bed after the period of time but before actually exiting the bed based on the number weight distribution signals; and
generating an alarm indicating imminent exit from the bed in response to the machine learning algorithm predicting that the individual will exit the bed.

13. The method according to claim 12, wherein the machine learning algorithm has been previously trained using ground truth data comprising in-bed weight distribution data and bed exit data obtained from a plurality of test subjects.
